SEQUENCE LISTING (11 sequences)

SEQ ID NO: 1 - DNA, 22 bp, Mus musculus
tgggatgaagggaagctaggac

SEQ ID NO: 2 - DNA, 21 bp, Mus musculus
ctttctgtcctatatcaactc

SEQ ID NO: 3 - DNA, 23 bp, Mus musculus
agagtgatcctgttcaacctgtg

SEQ ID NO: 4 - DNA, 27 bp, Escherichia coli
tgcgaggccagaggccacttgtgtagc

SEQ ID NO: 5 - DNA, 21 bp, Mus musculus
cttctttcttcgtcatgaacg

SEQ ID NO: 6 - DNA, 22 bp, Mus musculus
attcaacccccttgctttgctg

SEQ ID NO: 7 - DNA, 21 bp, Mus musculus
cccatcttctcctgaacgcca

SEQ ID NO: 8 - DNA, 4834 bp, Homo sapiens
gagccccgcatcccgccgcagctgccgcctcgccgcggccgggccggagagcacggcggcgggagcgcggccttaggaggcggccggagcggtgggcacagctcggcgcggagcgtcctgtcaggcggcggccgagggcgtcgcggactctccccgcgatgatgccgatgatattaactgttttcttgagcaacaatgaacagattttaacagaagttcctataacaccggaaacaacctgtcgagatgttgtagaattttgcaaggaacctggagaaggcagctgccatttagctgaagtgtggaggggaaatgaacgtcccataccctttgatcatatgatgtacgaacatcttcagatatggggtccacggagggaagaagtgaaatttttccttcgacacgaggactccccaactgagaacagtgaacaaggtggccgtcagacccaagagcaacgaactcagagaaatgtaataaatgtacctggagataaacgtactgaatatggggttgggaatccacgtgttgaacttaccctctcagagctccaagatatggcagctaggcaacagcagcagattgaaaatcagcagcagatgttggttgccaaggaacagcgtttacattttctaaagcaacaggagcgccgtcagcagcagtctatttctgaaaatgaaaagcttcagaaattgaaagaacgagttgaagcccaggagaacaagctgaagaaaattcgtgcaatgagaggacaagtcgactacagcaaaatcatgaacggcaatctgtctgctgaaatagaaaggttcagtgccatgttccaggaaaagaagcaggaagtacagactgcaattttaagggttgatcagcttagtcagcaattggaagatttaaagaaaggaaaactgaatgggttccagtcttacaatggcaaattgacgggaccagcggcggtggagttaaaaagactgtaccaagaactacagattcgtaaccaacttaaccaggaacaaaattcaaaacttcagcagcagaaggaactcttaaataagcgcaacatggaggtggccatgatggacaagcgaatcagtgaactgcgtgaacgtctctatgggaaaaaaattcagctgaaccgtgtgaatggcacgtcatcaccacagtcccctctgagcacatcgggcagggtcgctgctgtggggccttatatccaggttcccagtgccggaagctttcctgtgctgggggaccctataaagccccagtctctcagtattgcctcaaatgctgctcatggaagatccaaatccgctaatgatggaaactggccaacattaaaacagaattctagctcttccgtgaaaccagtgcaggtggccggtgcagactggaaggatccgagcgtggaggggtctgtcaagcagggcactgtctccagccagcctgtgcccttctcagcactgggacccacggagaagccgggcatcgagattggtaaagtgccacctcccatcccgggtgtaggcaagcagctgcctccaagctatgggacatacccaagtcctacacctctgggtcctgggtcgacaagctccctggaaaggaggaaggaaggcagcttgcccaggcccagtgcaggcctgccaagtcgacagaggcccaccctgctgcccgccacaggcagcaccccccagccaggctcctcacaacagattcagcagaggatttccgtaccgccaagtcccacgtacccgccagcgggaccacctgcatttccagctggggacagcaagcctgaactcccactgacagtggccattaggcctttcctggctgataaagggtcaaggccacagtctcccaggaaaggaccccagacagtgaattcaagttccatatactccatgtacctccagcaagccacaccacctaagaattaccagccggcagcacacagcgccttaaataagtcagttaaagcagtgtatggtaagcccgttttaccttcgggttcaacctctccatcgccgctgccgtttcttcacgggtcactgtccacgggcacaccacagcctcagccaccttcagaaagtactgagaaagagcctgagcaggatggccccgccgcccccgcagatggcagcaccgtggagagcctgccacggccactcagccccaccaagctcacgcccatcgtgcattcgccactgcgctaccagagtgatgcagacctggaggccctccgcaggaagctggccaacgcgccccggcccctgaaaaagcgcagctccatcacagagcccgagggccccggcgggcccaacatccagaagctgctgtaccagcgcttcaacaccctggccggtggcatggagggcacccctttctaccagcccagcccctcccaggacttcatgggcaccttggccgatgtggacaatggaaacaccaatgccaatggaaacctggaagagctcccccctgcccagcccacagccccactccccgctgagcctgccccgtcatcagatgccaatgataatgagttaccttcccccgaaccagaggagctcatctgtccccaaaccacccaccaaactgccgagccggcagaggacaataacaacaacgtggccacggtccccaccacggagcagatcccgagtcctgtggctgaggccccatctccaggggaagagcaggtccctccagcacctcttccccctgccagccaccctcctgccacctccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgatgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcctgaacttccttttggagcaccgcatggtcttgccagctaccaggagccacttaagagattattgtgctgttttccaggaaagctgcagctagaaaatggtcttaatggtgctcactttagcagacagcgtccacaatgtgaatcctacagtttccaggtgaggccctttctccagtttgcccattaactgggagaggtactttcgcctccaaggactgaattttgccaattactataaatccaaataaatacccactttcaaaacacccacccctcttgccattaagaagtcccataacccccggttggttgccagtgaagacagaagctcttactgacttggccccgaggccatcaccccctccagcagtgaacactgtccgccgctgtgaggcctgctcccctgcgaccgccctgccccccgtcaccgaatcggacactcatcctttctcacacttcccacacatgatccttcttcccttcatcaccaaaggagcctctgtatggaaacatgtccagtgttgctgcccagtgtgtatgcctcccagtacccactctgctcggccgccttgggggttccgcttcctgttccagttcacctaaaggctgattgtgcaggcccagcactgtggctggactgccgcgccacgggcaccaggacccctaagaccaagtgacaactgggagagcctcagcatatactcttctcctccgatctcacagcctgtcatgctgctcagtgtggttctcacccctgcaagctcaaattcagttccctgaatggagtcaggtgctggaggccgtggcagcggagggtggttggggttggggctgggggtggactggtgtgagggcagaccagggccaggtagacggggctgtttggtgcctgaaggatggcagacgcctggtgtcaggaggggccgccaccaaggagcagcagctggggcagaggagctggggtcaggggccacccctctctgccgatctccctgcctgggctggctgtgaggccacctttgtcccaggcccagcctcaaggcaaggagggcgcttcactgaggtgtgaattgtacgtacaggctttttatataccaaaagtattttttgactagaccattcaaagctacccgaactatgttggaaatttttttttttctcattaaaatacaggcccttaggctctatttttcatgtatgagtcgtgtgtaatttatgtaaaaatgtgtgtacagactcactgatgcagcactgtagcccatcaccttggagcactgactgtacatagtgtggtgaagaaaagtgaacgcccttgtagagcagcccgaccacaggagcatggccgctgccagcccagacgctgctgacgctgtgtaaatgtgcacaataaacccgtctcaccccgg

SEQ ID NO: 9 - DNA, 4402 bp, Homo sapiens
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcgtctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctccccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 10 - protein, 1090 aa, Homo sapiens
MMPMILTVFLSNNEQILTEVPITPETTCRDVVEFCKEPGEGSCHLAEVWRGNERPIPFDHMMYEHLQIWGPRREEVKFFLRHEDSPTENSEQGGRQTQEQRTQRNVINVPGDKRTEYGVGNPRVELTLSELQDMAARQQQQIENQQQMLVAKEQRLHFLKQQERRQQQSISENEKLQKLKERVEAQENKLKKIRAMRGQVDYSKIMNGNLSAEIERFSAMFQEKKQEVQTAILRVDQLSQQLEDLKKGKLNGFQSYNGKLTGPAAVELKRLYQELQIRNQLNQEQNSKLQQQKELLNKRNMEVAMMDKRISELRERLYGKKIQLNRVNGTSSPQSPLSTSGRVAAVGPYIQVPSAGSFPVLGDPIKPQSLSIASNAAHGRSKSANDGNWPTLKQNSSSSVKPVQVAGADWKDPSVEGSVKQGTVSSQPVPFSALGPTEKPGIEIGKVPPPIPGVGKQLPPSYGTYPSPTPLGPGSTSSLERRKEGSLPRPSAGLPSRQRPTLLPATGSTPQPGSSQQIQQRISVPPSPTYPPAGPPAFPAGDSKPELPLTVAIRPFLADKGSRPQSPRKGPQTVNSSSIYSMYLQQATPPKNYQPAAHSALNKSVKAVYGKPVLPSGSTSPSPLPFLHGSLSTGTPQPQPPSESTEKEPEQDGPAAPADGSTVESLPRPLSPTKLTPIVHSPLRYQSDADLEALRRKLANAPRPLKKRSSITEPEGPGGPNIQKLLYQRFNTLAGGMEGTPFYQPSPSQDFMGTLADVDNGNTNANGNLEELPPAQPTAPLPAEPAPSSDANDNELPSPEPEELICPQTTHQTAEPAEDNNNNVATVPTTEQIPSPVAEAPSPGEEQVPPAPLPPASHPPATSTNKRTNLKKPNSERTGHGLRVRFNPLALLLDASLEGEFDLVQRIIYEVEDPSKPNDEGITPLHNAVCAGHHHIVKFLLDFGVNVNAADSDGWTPLHCAASCNSVHLCKQLVESGAAIFASTISDIETAADKCEEMEEGYIQCSQFLYGVQEKLGVMNKGVAYALWDYEAQNSDELSFHEGDALTILRRKDESETEWWWARLGDREGYVPKNLLGLYPRIKPRQRTLA

SEQ ID NO: 11 - protein, 1128 aa, Homo sapiens
MMPMFLTVYLSNNEQHFTEVPVTPETICRDVVDLCKEPGESDCHLAEVWCGSERPVADNERMFDVLQRFGSQRNEVRFFLRHERPPGRDIVSGPRSQDPSLKRNGVKVPGEYRRKENGVNSPRMDLTLAELQEMASRQQQQIEAQQQLLATKEQRLKFLKQQDQRQQQQVAEQEKLKRLKEIAENQEAKLKKVRALKGHVEQKRLSNGKLVEEIEQMNNLFQQKQRELVLAVSKVEELTRQLEMLKNGRIDSHHDNQSAVAELDRLYKELQLRNKLNQEQNAKLQQQRECLNKRNSEVAVMDKRVNELRDRLWKKKAALQQKENLPVSSDGNLPQQAASAPSRVAAVGPYIQSSTMPRMPSRPELLVKPALPDGSLVIQASEGPMKIQTLPNMRSGAASQTKGSKIHPVGPDWSPSNADLFPSQGSASVPQSTGNALDQVDDGEVPLREKEKKVRPFSMFDAVDQSNAPPSFGTLRKNQSSEDILRDAQVANKNVAKVPPPVPTKPKQINLPYFGQTNQPPSDIKPDGSSQQLSTVVPSMGTKPKPAGQQPRVLLSPSIPSVGQDQTLSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPEEPNPEAPHVLDVYLEEYPPYPPPPYPSGEPEGPGEDSVSMRPPEITGQVSLPPGKRTNLRKTGSERIAHGMRVKFNPLALLLDSSLEGEFDLVQRIIYEVDDPSLPNDEGITALHNAVCAGHTEIVKFLVQFGVNVNAADSDGWTPLHCAASCNNVQVCKFLVESGAAVFAMTYSDMQTAADKCEEMEEGYTQCSQFLYGVQEKMGIMNKGVIYALWDYEPQNDDELPMKEGDCMTIIHREDEDEIEWWWARLNDKEGYVPRNLLGLYPRIKPRQRSLA

(19) United States
(12) Patent Application Publication
(10) Pub. No.: US 2006/0127401 A1
(43) Pub. Date: Jun. 15, 2006

(54) Screening method

(76) Inventor: Xin Lu, London (GB)

Correspondence Address: KLARQUIST SPARKMAN, LLP, 121 SW SALMON STREET, SUITE 1600, PORTLAND, OR, 97204, US

(21) Appl. No.: 10/530,450

(22) PCT Filed: Oct. 3, 2003
(86) PCT No.: PCT/GB03/04258

§ 371 Date: Dec. 22, 2005

(30) Foreign Application Priority Data
Oct. 7, 2002 (GB) 0223187.6

(51) Int. Cl. A61K 48/00; C12Q 1/68; G01N 33/567; A61K 39/395; G01N 33/53

(52) U.S. Cl. 424/145.1; 514/44; 435/6; 435/7.2

(57) ABSTRACT

The invention relates to a screening method to determine the susceptibility of a mammal, preferably a human, to abnormal development of the nervous system and including therapeutic methods and compositions for the treatment of neurodegenerative conditions which result in abnormal expression of a family of polypeptides which induce the apoptotic function of p53.

The invention relates to a screening method to determine the susceptibility of a mammal, preferably a human, to abnormal development of the nervous system and including therapeutic methods and compositions for the treatment of neurodegenerative conditions which result in abnormal expression of a family of polypeptides which induce the apoptotic function of p53.

Apoptosis, or programmed cell death, is a process by which multi-cellular organisms regulate cell number and differentiation. The process is regulated by factors which either induce or prevent apoptosis. Inducers of apoptosis include Bcl-2 family members, caspase family members and their associated factors Apaf-1 and Fadd. Caspases are synthesised as proenzymes which become activated after proteolytic cleavage. The active caspase then induces many of the morphological and biochemical changes associated with apoptosis. Mitochondria play a pivotal role in the activation process through the release of pro-apoptotic factors such as cytochrome c, AIF and Diablo. The release from mitochondria is controlled by the Bcl-2 family of proteins; (e.g. Bcl-2 and Bcl-x1 inhibit release; Bax and Bak induce release). WO9953051 discloses a cytokine dependent protein p21 which has pro-apoptotic activity. p21 is expressed in a cytokine dependent manner in myeloid/erthyroid cells. These cells are dependent on IL-3 for growth and in the absence of IL-3 the translation of p21 is induced resulting in apoptosis and cell death. p21 is a cytoplasmic protein which translocates to the outer mitochondrial membrane to induce pro-apoptotic activities.

Tumour suppressor proteins also have pro-apoptotic activities.

Tumour suppressor genes encode proteins which function to inhibit cell growth or division and are therefore important with respect to influencing proliferation and maintaining growth and differentiation of normal cells. Mutations in tumour suppressor genes result in abnormal cell-cycle progression whereby the normal cell-cycle check points which arrest the cell-cycle, when, for example, DNA is damaged, are ignored and damaged cells divide uncontrollably. The products of tumour suppressor genes function in all parts of the cell (e.g. cell surface, cytoplasm, nucleus) to prevent the passage of damaged cells through the cell- cycle (i.e. G1, S, G2, M and cytokinesis).

Arguably the tumour suppressor gene which has been the subject of the most intense research is p53. p53 encodes a protein which functions as a transcription factor and is a key regulator of the cell division cycle. It was discovered in 1978 as a protein shown to bind with affinity to the SV40 large T antigen. The p53 gene encodes a 393 amino acid polypeptide with a molecular weight of 53 kDa. Genes regulated by the transcriptional activity of p53 contain a p53 recognition sequence in their 5′ regions. These genes are activated when the cellular levels of p53 are elevated due to, for example DNA damage. Examples of genes which respond to p53 include, mdm2, Bax and PIG-3. Bax and PIG-3 are involved in one of the most important functions of p53, the induction of apoptosis.

In our co-pending application WO02/12325 we disclose, amongst other things, a new family of pro-apoptotic polypeptides which regulate the apoptotic activity of p53. We herein describe the involvement of this family in the development of the central nervous system (CNS) in mammals. Neonatal mice which are homozygous null for the ASPP 2 gene have abnormal development of the CNS. The cells of the CNS have abnormal patterns of division which results in abnormalities, particularly in the brain and retina, of developing neonatal mice. This is suggestive of the need for programmed cell death in the differentiation of the CNS and the involvement of the ASPP 2 family in regulating this process.

According to an aspect of the invention there is provided a method for the detection of a polypeptide in a cell or tissue sample which sample comprises a nerve cell or a nerve progenitor cell and wherein said polypeptide is a polypeptide which induces the apoptotic function of p53.

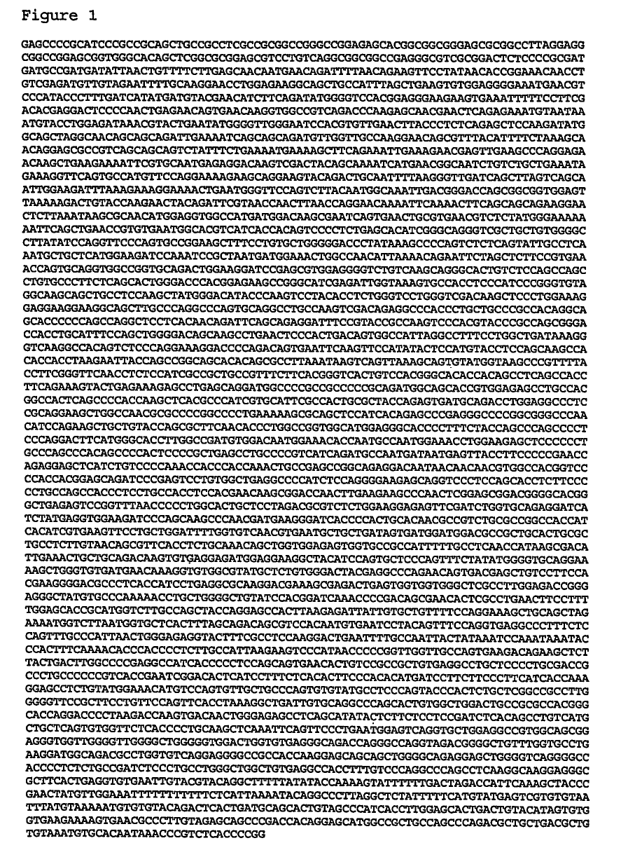

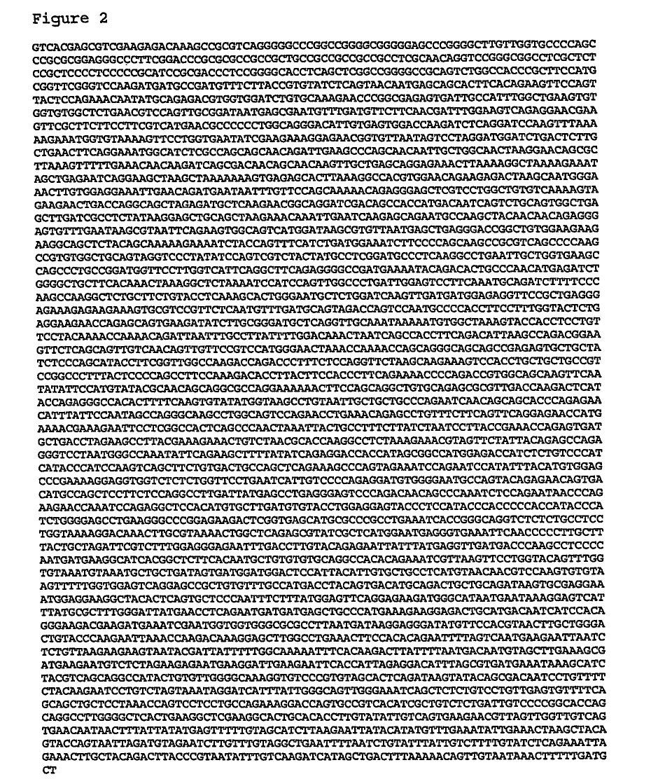

In a preferred method of the invention said polypeptide is selected from the group consisting of:
a) a polypeptide encoded by a nucleic acid molecule as represented by the sequence shown in FIGS. 1 or 2;
b) a polypeptide encoded by a nucleic acid molecule which hybridises to the nucleic acid molecule in (a); or
c) a polypeptide encoded by a nucleic acid molecule which is degenerate because of the genetic code to the nucleic acid molecule represented in (a) and (b); said method comprising the steps of;
   i) providing a sample comprising a nerve cell or a nerve cell progenitor cell;
   ii) contacting said sample with an agent which binds said polypeptide;
   iii) detecting the presence of said polypeptide in said cell sample.

In a preferred method of the invention said polypeptide is encoded by a nucleic acid molecule which hybridises under stringent hybridisation conditions to the nucleic acid sequence as represented in FIGS. 1 or 2. Preferably said nucleic acid is represented by the nucleic acid sequence in FIGS. 1 or 2.

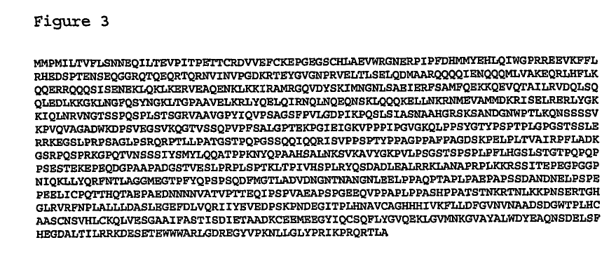

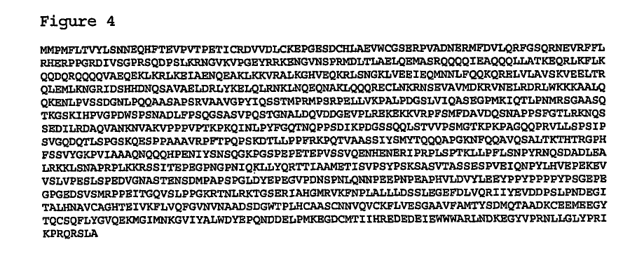

In a preferred embodiment of the invention said polypeptide is represented by the amino acid sequences in FIGS. 3 or 4 wherein said polypeptide is altered by addition, deletion or substitution of at least one amino acid residue.

In a preferred method of the invention said agent is an antibody which binds said polypeptide; preferably a polyclonal antibody.

In a further preferred method of the invention said antibody is a monoclonal antibody.

In a yet further preferred method of the invention said antibody is provided with means which enable the detection, either directly or indirectly, of the antibody bound to said polypeptide.

In a preferred method of the invention said detection means is selected from the group consisting of: an enzyme; a isotope label or a fluorescent label.

In an alternative preferred method of the invention said method is the detection of a nucleic acid molecule which encodes said polypeptide.

In an alternative preferred method of the invention said agent is a nucleic acid molecule adapted to anneal to said nucleic acid molecule which encodes said polypeptide.

In a preferred method of the invention said nucleic acid molecule is at least one oligonucleotide molecule. Preferably a pair of oligonucleotide molecules adapted to bind said nucleic acid molecule which is to be detected. Preferably said method is a polymerase chain reaction method.

According to a further aspect of the application there is provided the use of a polypeptide selected from the group consisting of:
i) a polypeptide encoded by a nucleic acid molecule as represented by the sequence shown in FIGS. 1 or 2;
ii) a polypeptide encoded by a nucleic acid molecule which hybridises to the nucleic acid molecule in (i); or
iii) a polypeptide encoded by a nucleic acid molecule which is degenerate because of the genetic code to the nucleic acid molecule represented in (i) and (ii).
   for use in the manufacture of a medicament for use in the treatment of neurodegenerative diseases which result from a deficiency in said polypeptide.

In a preferred embodiment of the invention said polypeptide is represented by the amino acid sequence presented in FIGS. 3 or 4 wherein said sequence has been modified by addition, deletion or substitution of at least one amino acid residue.

We disclose that mice which do not express ASPP family members show abnormal neural development in the neonatal CNS and retina as a result of uncontrolled cell division. This strongly implicates ASPP induced apoptosis in the development of the CNS. In addition there are a number of neurodegenerative diseases in which apoptosis is implicated. For example, Alzheimer's disease, Parkinson's disease, and multiple sclerosis. It is possible that inappropriate expression of ASPP family members in these conditions could result in premature cell death of neurones. This theory is readily testable by methods established in the art.

In a preferred embodiment of the invention said polypeptide is encoded by a nucleic acid molecule. Preferably said nucleic acid molecule is part of a vector adapted for gene therapy.

The invention also contemplates gene therapy of neurodegenerative diseases. The procedure for performing ex vivo gene therapy is outlined in U.S. Pat. No. 5,399,346 and in exhibits submitted in the file history of that patent, all of which are publicly available documents. In general, it involves introduction in vitro of a functional copy of a gene into a cell(s) of a subject which contains a defective copy of the gene, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy using vectors such as adenovirus, retroviruses, herpes virus, and targeted liposomes also is contemplated according to the invention.

According to a further aspect of the application there is provided the use of an antagonist which interacts with a polypeptide selected from the group consisting of:
i) a polypeptide encoded by a nucleic acid molecule as represented by the sequence shown in FIGS. 1 or 2;
ii) a polypeptide encoded by a nucleic acid molecule which hybridises to the nucleic acid molecule in (i); or
iii) a polypeptide encoded by a nucleic acid molecule which is degenerate because of the genetic code to the nucleic acid molecule represented in (i) and (ii).
   for use in the manufacture of a medicament for use in the treatment of neurodegenerative diseases which result from abnormal expression of said polypeptide.

In a preferred embodiment of the invention said polypeptide is represented by the amino acid sequence presented in FIGS. 3 or 4 wherein said sequence has been modified by addition, deletion or substitution of at least one amino acid residue.

In a further preferred method of the invention said disease is selected from the group consisting of: Alzheimer's disease; Parkinson's disease; multiple sclerosis; retinopathies.

In a preferred embodiment of the invention said antagonist is an antibody or antibody part which binds said polypeptide. Preferably said antibody is a monoclonal antibody or binding part thereof.

Antibodies, also known as immunoglobulins, are protein molecules which usually have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability; it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the ‘constant’ regions of the H chains, i.e., IgG1, IgG2, IgG3 and IgG4). Further detail regarding antibody structure and their various functions can be found in, Using Antibodies: A laboratory manual, Cold Spring Harbour Laboratory Press.

In a preferred embodiment of the invention said fragment is a Fab fragment.

In a further preferred embodiment of the invention said antibody is selected from the group consisting of: F(ab′)₂, Fab, Fv and Fd fragments; and antibodies comprising CDR3 regions.

Preferably said fragments are single chain antibody variable regions (scFV's) or domain antibodies. If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

A modified antibody, or variant antibody, and reference antibody, may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Most highly preferred are variants which show enhanced biological activity.

Preferably said antibody is a humanised or chimeric antibody.

A chimeric antibody is produced by recombinant methods to contain the variable region of an antibody with an invariant or constant region of a human antibody.

A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In an alternative preferred embodiment of the invention said antagonist is a nucleic acid molecule.

In a preferred embodiment of the invention said nucleic acid molecule is selected from the group consisting of an antisense molecule or an inhibitory RNA molecule designed with reference to FIGS. 1 or 2.

As used herein, the term “antisense molecule” or “antisense” describes an oligonucleotide that is an oligoribonucleotide, oligodeoxyribonucleotide, modified oligoribonucleotide, or modified oligodeoxyribonucleotide which hybridises under physiological conditions to DNA comprising a particular gene or to an mRNA transcript that gene and, thereby, inhibits the transcription of that gene and/or the translation of that mRNA. The antisense molecules are designed so as to interfere with transcription or translation of a target gene upon hybridisation with the target gene or transcript. Those skilled in the art will recognise that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridise substantially more to the target sequence than to any other sequence in the target cell under physiological conditions. Based upon the ASPP-2 nucleic acid sequences provided herein, or upon allelic or homologous genomic and/or cDNA sequences, one of skill in the art can easily choose and synthesise any of a number of appropriate antisense molecules for use in accordance with the present invention. For example, a “gene walk” comprising a series of oligonucleotides of 15-30 nucleotides spanning the length of ASPP-2 nucleic acid can be prepared, followed by testing for inhibition of the corresponding ASPP-2 expression. Optionally, gaps of 5-10 nucleotides can be left between the oligonucleotides to reduce the number of oligonucleotides synthesised and tested.

In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. Finally, although ASPP1/ASPP-2 cDNA sequences are disclosed herein, one of ordinary skill in the art may easily derive the genomic DNA corresponding to these cDNAs. Thus, the present invention also provides for antisense oligonucleotides which are complementary to ASPP1/ASPP-2 genomic DNA. Similarly, antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognised methods which may be carried out manually or by an automated synthesiser. They also may be produced recombinantly by vectors.

A recent technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated.

Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

In a preferred embodiment of the invention there is provided a transcription cassette comprising an nucleic acid sequence operatively linked to a promoter which promoter transcribes said nucleic acid molecule to produce an antisense nucleic acid molecule, said sequence selected from the group consisting of:
i) a nucleic acid sequence, or part thereof, as represented in FIGS. 1 or 2;
ii) a nucleic acid sequence which hybridises to the sense sequence presented in FIGS. 1 or 2 and which encodes a polypeptide with anti-apoptotic activity.

In a preferred embodiment of the invention said cassette is part of a vector.

In a further preferred embodiment of the invention there is provided a transcription cassette comprising a nucleic acid molecule, or part thereof, selected from the group consisting of:
i) a nucleic acid molecule represented by the nucleic acid sequence in FIGS. 1 or 2;
ii) a nucleic acid molecule which hybridises to the sequence in (i) above and which encodes a polypeptide with anti-apoptotic activity, or
iii) a nucleic acid molecule which is degenerate because of the genetic code to the sequences defined in (i) and (ii) above; wherein said cassette is adapted such that both sense and antisense nucleic acid molecules are transcribed from said cassette.

In a preferred embodiment of the invention said cassette is provided with at least two promoters adapted to transcribe both sense and antisense strands of said nucleic acid molecule.

In a further preferred embodiment of the invention said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

In a preferred embodiment of the invention said first and second parts are linked by at least one nucleotide base.

In a preferred embodiment of the invention said first and second parts are linked by 2, 3, 4, 5, 6, 7, 8, 9 or at least 10 nucleotide bases.

In a further preferred embodiment of the invention the length of the RNAi molecule is between 100 bp-1000 bp. More preferably still the length of RNAi is selected from 100 bp; 200 bp; 300 bp; 400 bp; 500 bp; 600 bp; 700 bp; 800 bp; 900 bp; or 1000 bp. More preferably still said RNAi is at least 1000 bp.

In an alternative preferred embodiment of the invention the RNAi molecule is between 15 bp and 25 bp, preferably said molecule is 21 bp.

In a preferred embodiment of the invention said cassette is part of a vector.

According to a further aspect of the invention there is provided method to screen for agents which modulate the activity of a polypeptide which induces the apoptotic function of p53 comprising the steps of:
i) providing a cell sample comprising a nerve cell or nerve progenitor cell;
ii) contacting said sample with an agent to be tested; and
iii) monitoring effect of said agent on the presence and/or activity of said polypeptide.

In a preferred method of the invention said polypeptide is selected from the group consisting of:
a) a polypeptide encoded by a nucleic acid molecule as represented by the sequence shown in FIGS. 1 or 2;
b) a polypeptide, encoded by a nucleic acid molecule which hybridises to the nucleic acid molecule in (a); or
c) a polypeptide encoded by a nucleic acid molecule which is degenerate to the nucleic acid molecule represented in (a) and (b).

In a further preferred method of the invention said agent is an antagonist of said polypeptide.

In an alternative preferred method of the invention said agent is an agonist of said polypeptide.

An embodiment of the invention will now be described by example only and with reference to the following figures:

FIG. 1 is a DNA sequence which encodes ASPP1;

FIG. 2 is a DNA sequence which encodes ASPP2;

FIG. 3 is the amino acid sequence of ASPP1;

FIG. 4 is the amino acid sequence of ASPP2;

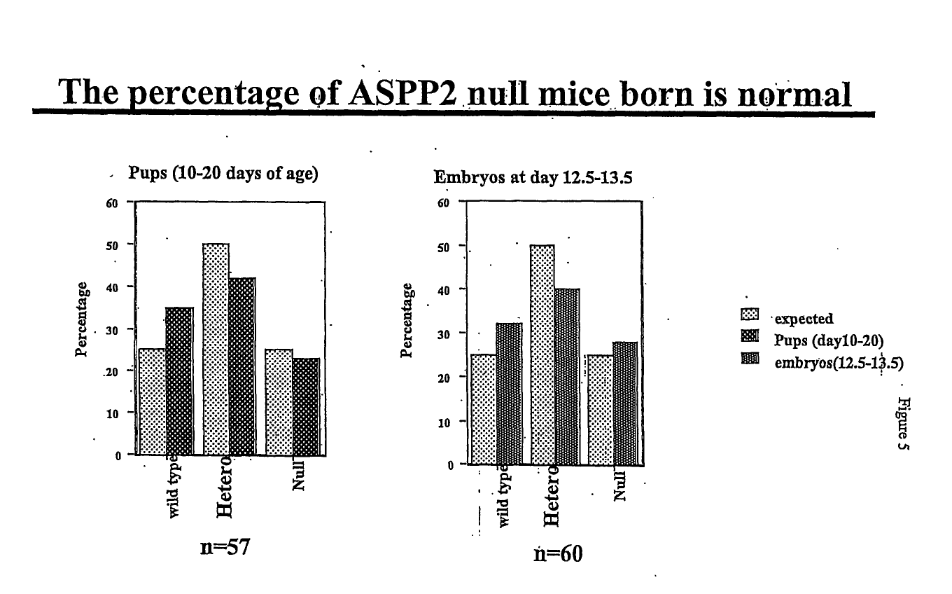

FIG. 5 illustrates that the percentage of ASPP2 null mice born is normal;

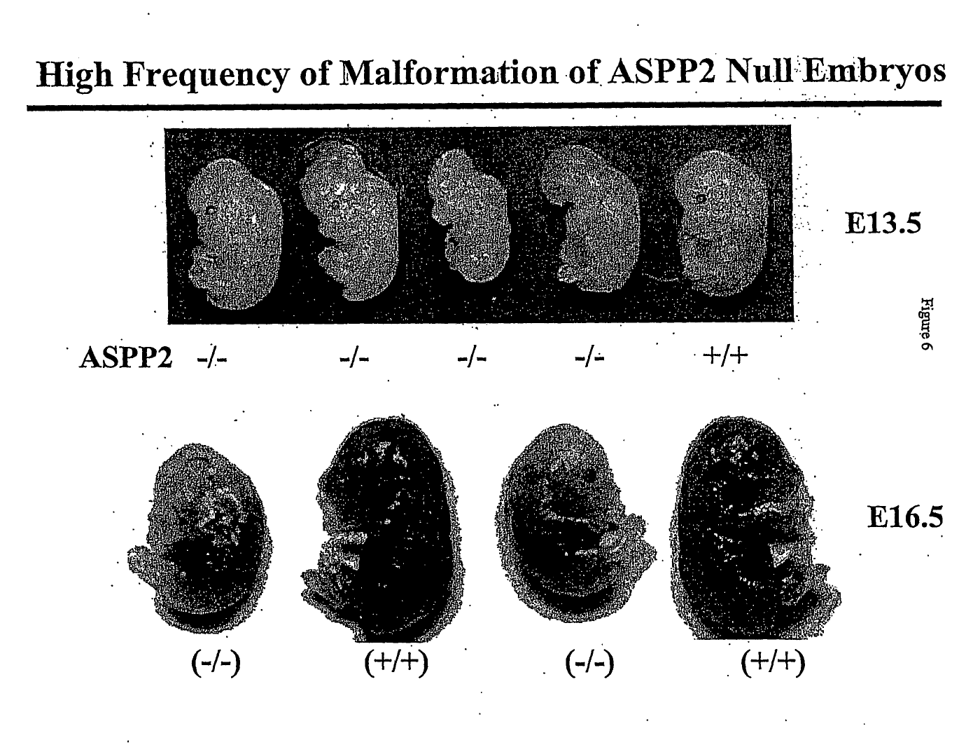

FIG. 6 illustrates the malformation of ASPP2 null embryos;

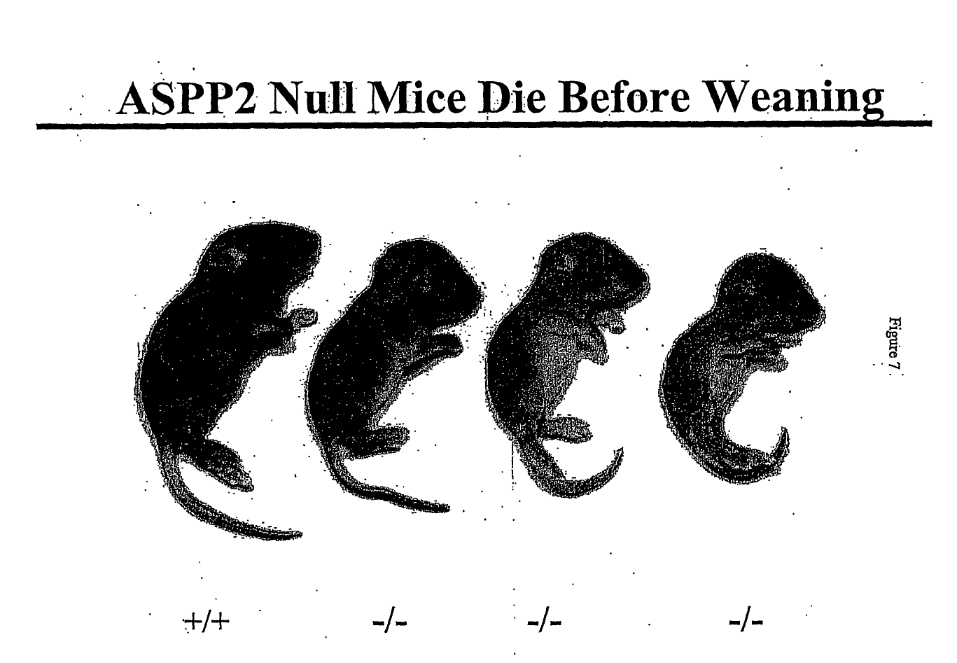

FIG. 7 illustrates that ASPP2 null mice die before weaning;

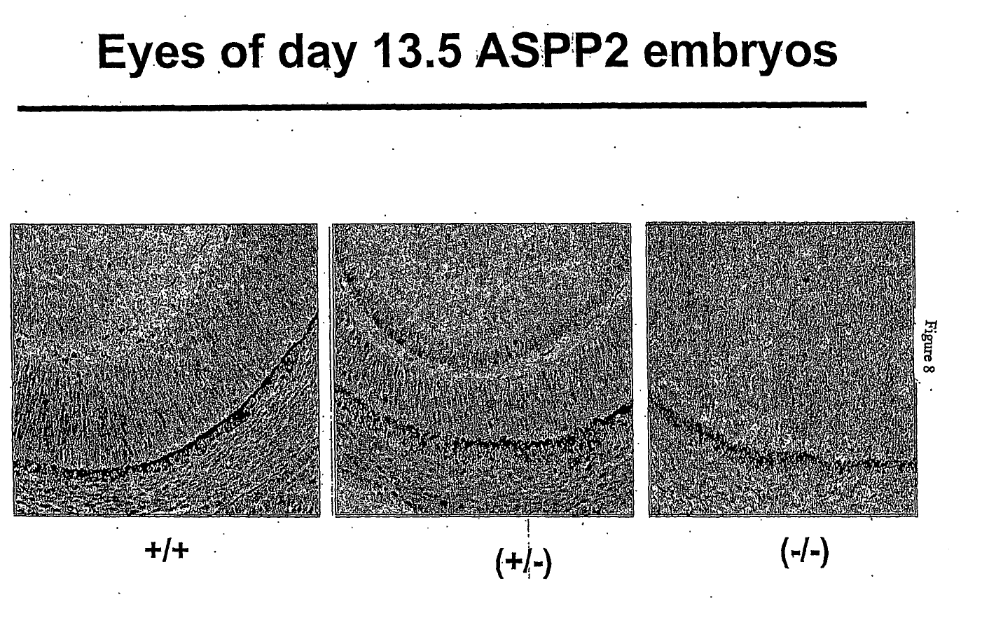

FIG. 8 illustrates eye malformation in 13.5 day embryos in ASPP2 (±) and ASPP2 (+/+) mice;

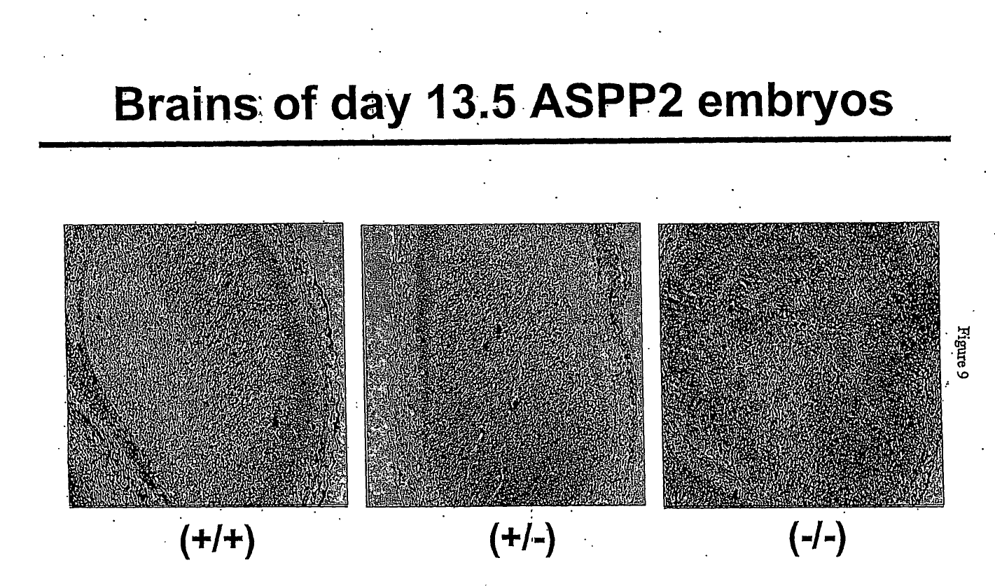

FIG. 9 illustrates brain malformation 13.5 day embryos in ASPP2 (±) and ASPP2 (+/+) mice;

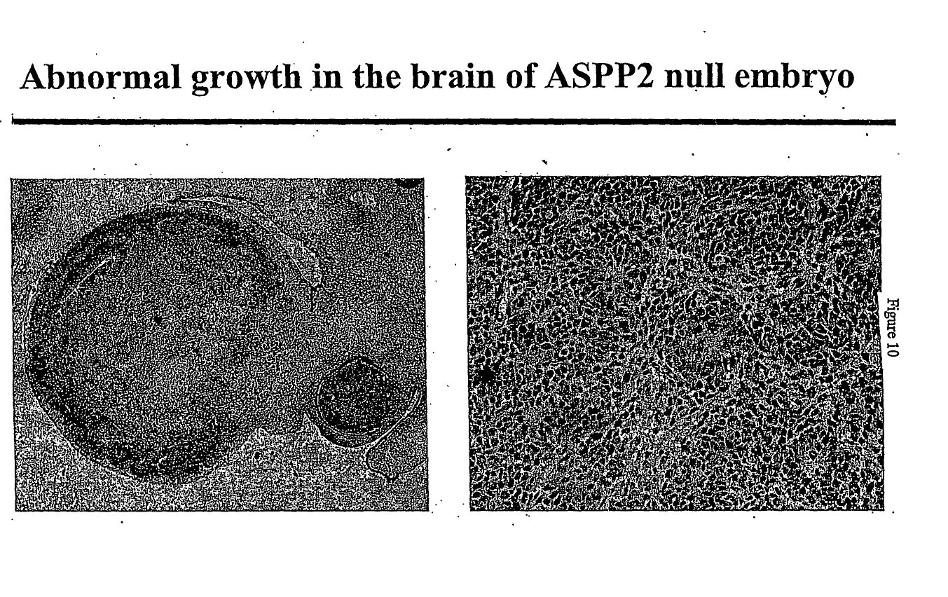

FIG. 10 illustrates abnormal cell growth in the brain of an ASPP2 null embryo;

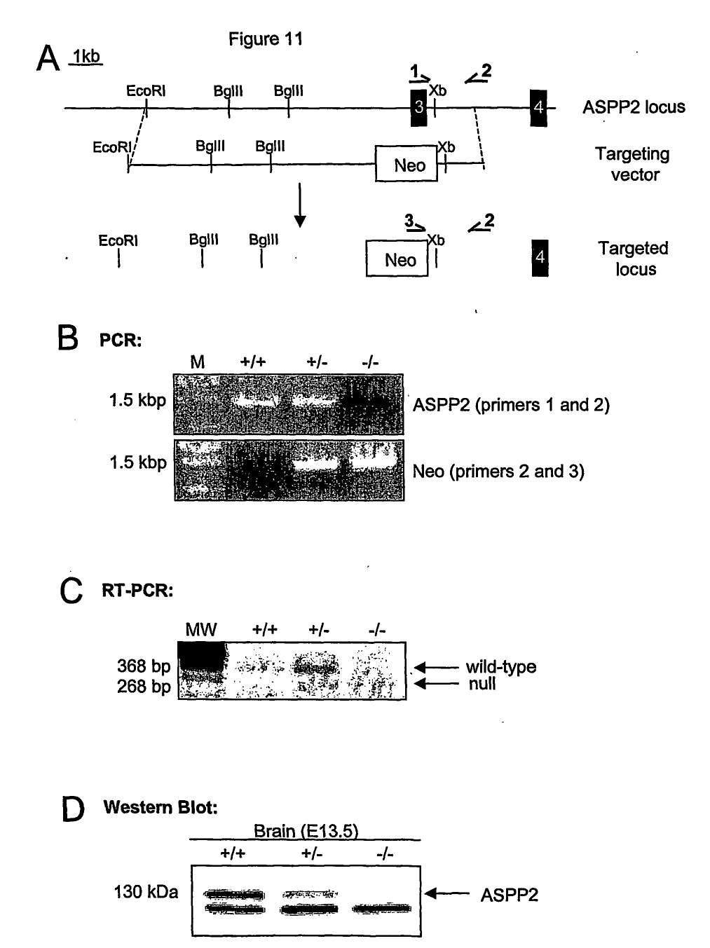

FIG. 11A illustrates a map of the ASPP2 locus and targeting vector; FIG. 11B is a PCR reaction confirming the genotype of homozygous and heterozygous mice deleted for ASPP2; FIG. 11C is an RT PCR reaction confirming the lack of expression of ASPP2 in ASPP2 knock-out mice; and FIG. 11D is a western blot confirming the lack of expression of ASPP2 protein in ASPP2 knock-out mice;

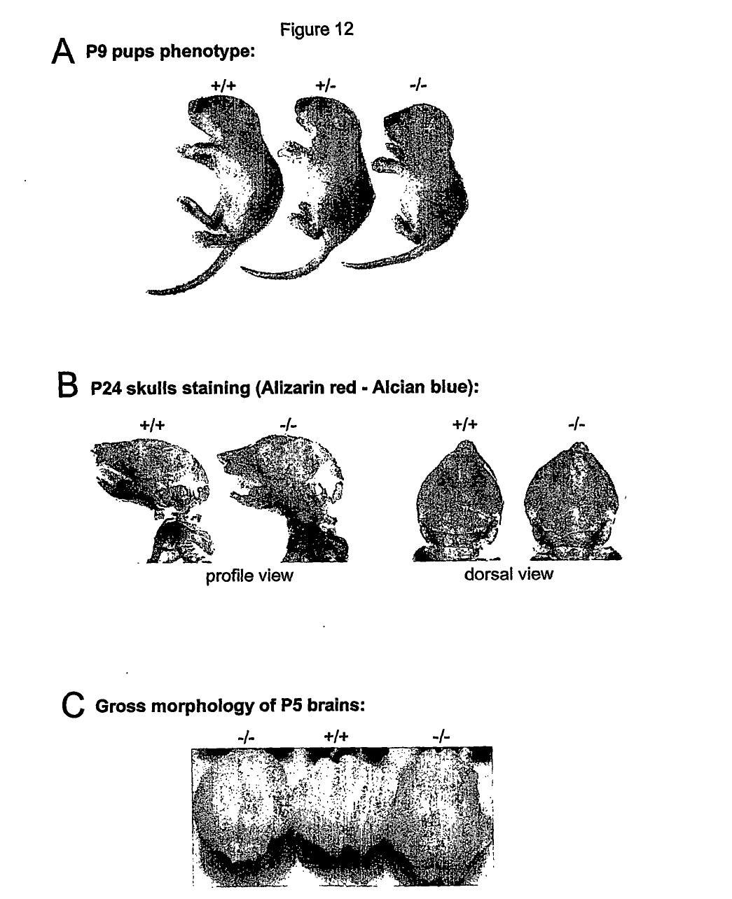

FIG. 12 illustrates the phenotype of ASPP2 knock-out mice;

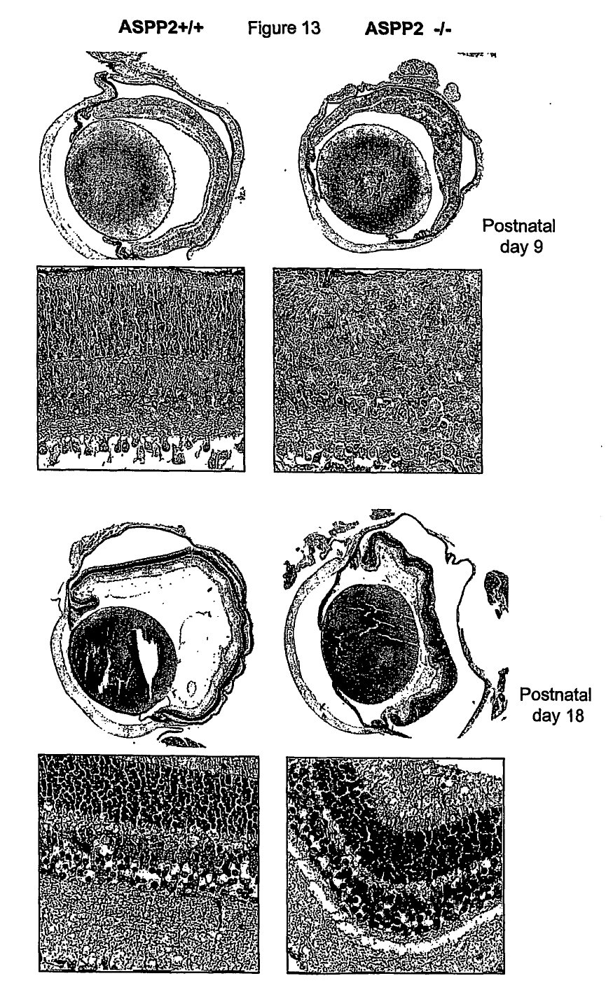

FIG. 13 illustrates the effect of ASPP2 genotype on retina morphology;

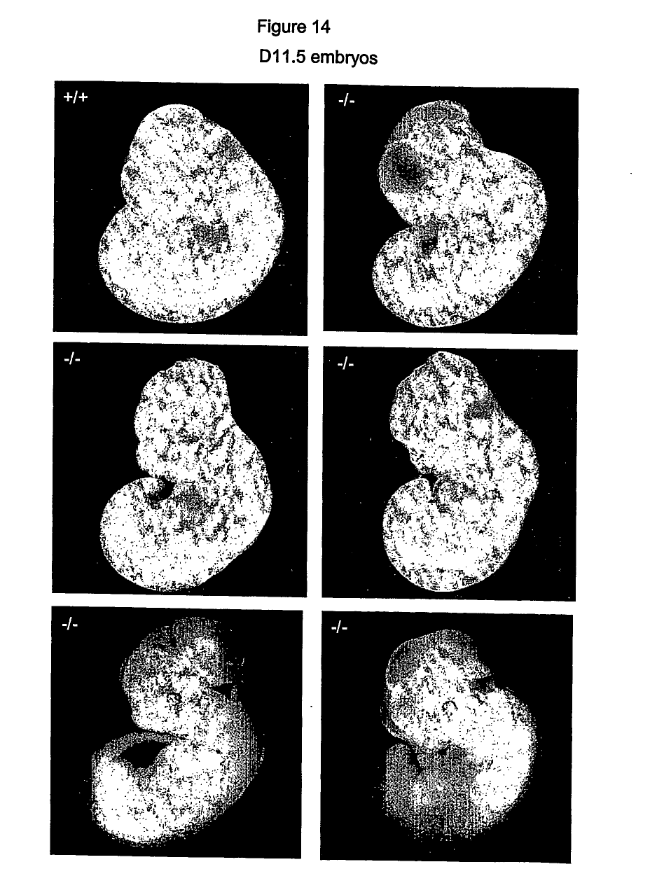

FIG. 14 illustrates the mouse brain phenotype in ASPP2 null mice;

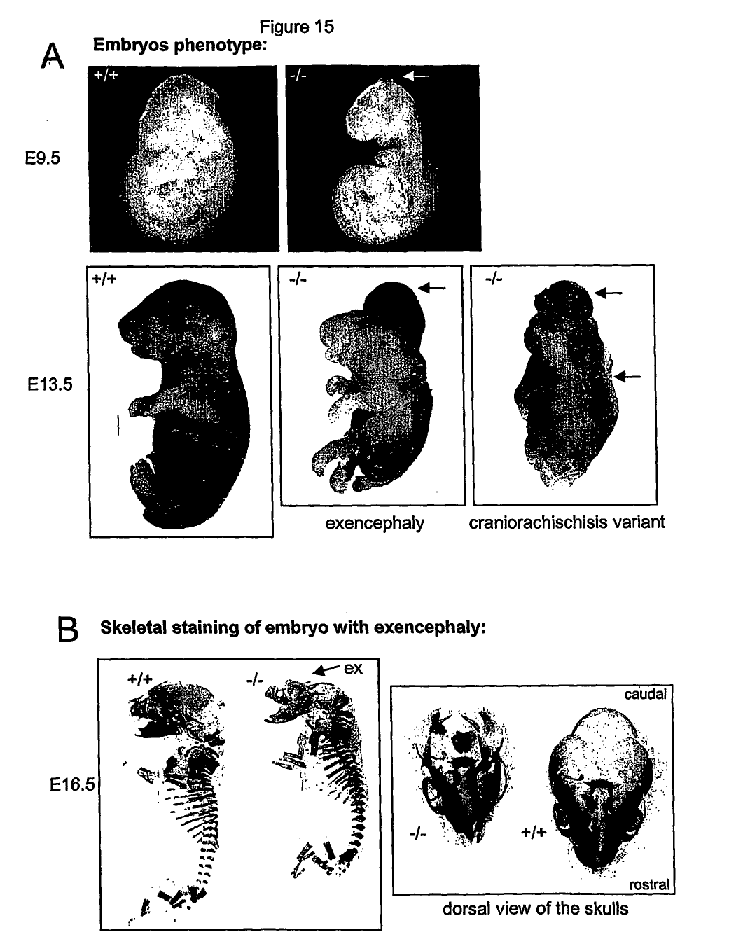

FIG. 15 illustrates the mouse skull phenotype in ASPP2 null mice;

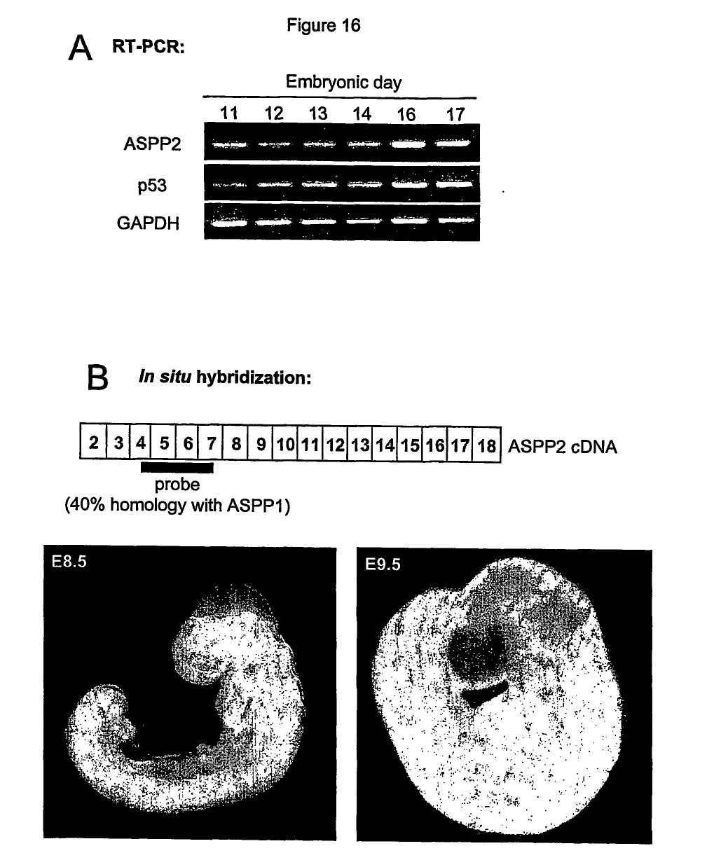

FIG. 16A illustrates RT PCR of RNA encoding ASPP2 during mouse embryonic development; FIG. 16B illustrates an in situ hybridisation to confirm when ASPP2 is expressed during mouse embryo development;

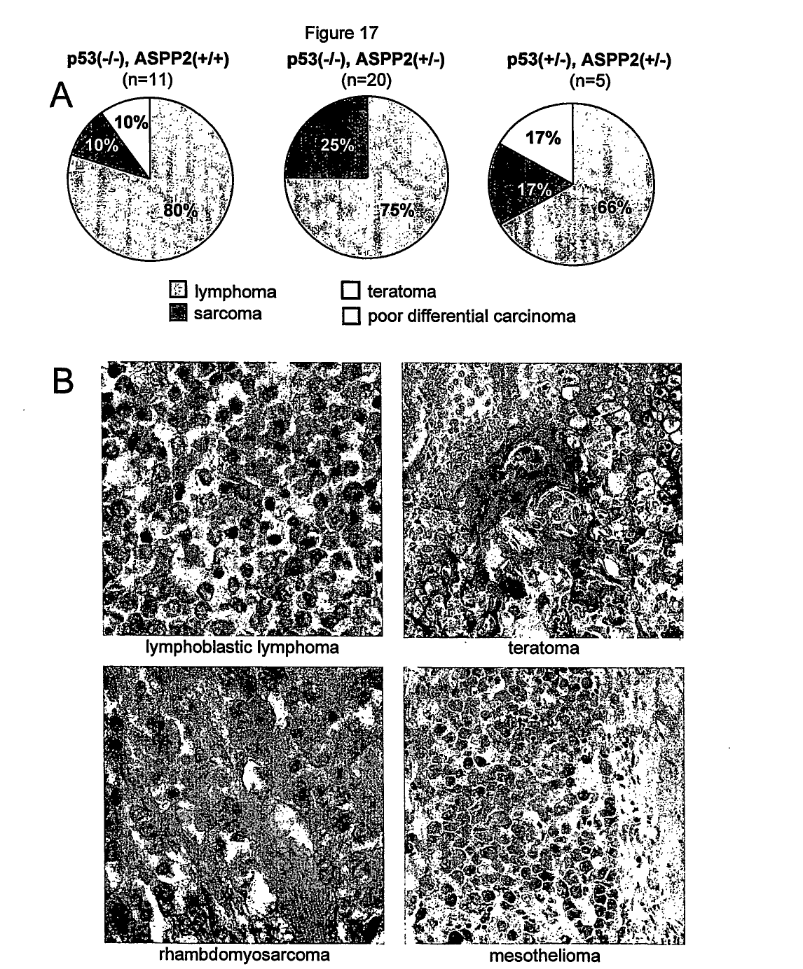

FIG. 17 illustrates the analysis of tumour generation in various p53/ASPP2 genotypes; and

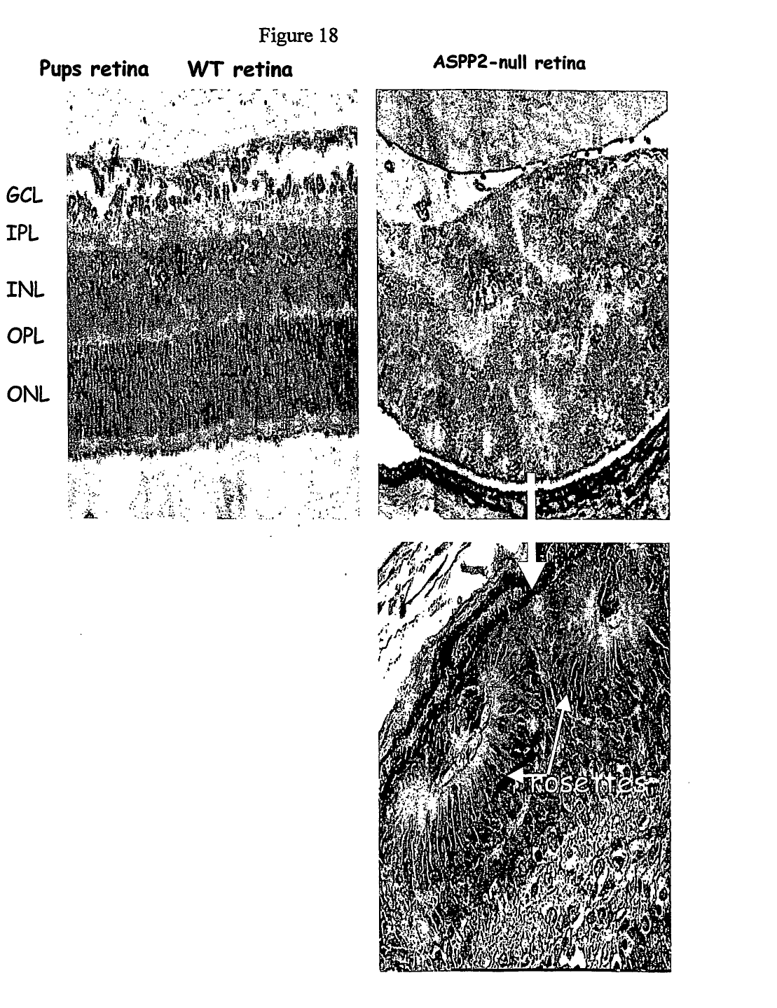

FIG. 18 illustrates the effect of a lack of ASPP2 and the effect this has on the development of retinoblastoma.

### MATERIALS AND METHODS

Targeted Disruption of the ASPP2 Gene.

The mouse P53BP2 gene was cloned from the 129 SVJ phage genomic library. Exons were mapped. The targeting vector was constructed by using 1.3 kb DNA fragment as the short arm, which was a PCR fragment from primers X23 to X22. Primer X23 is located about 130 bp downstream of putative exon 1 inside intron 1 with a sequence of 5′-TGGGATGAAGGGAAGCTAGGAC-3′. Primer X22 is located 1.4 kb further downstream of putative exon 1 inside intron 1 with a sequence of 5′-CTTTCTGTCCTATATCAACTC-3′. The long arm was made of a fragment from EcoRV to the end of the P53BP2 lambda genomic clone. In this knockout strategy, putative exon 1 and 2 kb upstream: sequence were replaced by the Neo gene cassette. Ten micrograms of the targeting vector was linearized by NotI and then transfected by electroporation of IT2 embryonic stem cells. After selection in G418, surviving colonies were expanded, and PCR analysis was performed to identify clones that had undergone homologous recombination. PCR was done using primer pair X25 and Neo1. Primer X25 is located 100 bp downstream of primer X22 with a sequence of 5′-AGAGTGATCCTGTTCAACCTGTG-3′. Primer Neo1 is located in the 5′-promoter region of the Neo gene cassette and has a sequence of 5′-TGCGAGGCCAGAGGCCACTTGTGTAGC-3′. The positive clones will give rise to a 1.5 kb PCR fragment. The correctly targeted ES cell lines were microinjected into C57BL/6J host blastocysts. The chimeric mice were generated and they gave germline transmission of the disrupted P53BP2 gene. To identify the wild-allele, primer pair X5 and X25 can be used. Primer X5 is located inside exon 1 with a sequence of 5′-CTTCTTTCTTCGTCATGAACG-3′. The PCR product should be around 1.5 kb. In the homozygous knockout mice, this PCR band would not be amplified.

RT-PCR Analysis of ASPP2 and p53 mRNA.

Total RNA was isolated from whole embryos by Trizol (GIBCO-BRL). Five micrograms of total RNA was reverse-transcribed using the superscript first-strand synthesis system (IN VITROGEN), and the resulting templates were subjected to a PCR reaction with ASPP2 specific primers (5′-ATTCAACCCCCTTGCTTTGCTG-3′ and 5′-CCCATCTTCTCCTGAACGCCA-3′) or primers specific for p53 or GAPDH.

Histology

Fetuses fixed in 10% buffered formalin were embedded in paraffin and cut in 4 μm sections. They were then stained with Haematoxilin and Eosin.

Tumour Analysis

Mice that developed visible tumours approximately 1 cm in diameter were killed and subjected to necropsy. Mice that did not develop visible tumours but became moribund were also killed and subjected to necropsy. In addition to tumours samples, tissues from brain, eyes, heart, lung, liver, kidney and testis were recovered. The mice were carefully examined for the presence of any other abnormalities. All the tissues were fixed in 10% buffered formalin, processed for histology and paraffin-embedded. Four-micrometers-thick paraffin sections were stained with haematoxilin and eosin.

### EXAMPLES

Generation of ASPP2 Null Mice.

To study the functional importance of ASPP2 in embryogenesis, we inactivated the murine ASPP2 gene. The ASPP2 gene was disrupted in mouse IT2 embryonic stem (ES) cells using a targeting vector in which the exon 3 was deleted (FIG. 11A). The linearized targeting vector was electroporated into ES cells and G418-resistant colonies were found to be heterozygous for the mutation at the ASPP2 locus by PCR (FIG. 11B). Heterozygous ES clones were independently injected into C57BL/6J blastocysts and gave rise to germ line-transmitting chimeric mice that were crossed into a C57B1/6 background. The resulting heterozygous mice appeared normal and fertile. They were then used to breed ASPP2 homozygous mutant progeny. To test for the generation of true null mutants, the ASPP2 mRNA and protein levels were analysed by RT-PCR and western blot. As expected, although the truncated ASPP2 mRNA transcript was detected (FIG. 11C), the WT ASPP2 protein was not expressed (FIG. 11D) in null embryos compared to wild-type littermates.

ASPP2 Null Mice have Severe Brain Malformations and Die Around Birth.

Of more than 600 offsprings derived from heterozygous matings, only 6.62% homozygous ASPP2 mutants were identified (table 1). Moreover, all died within 2 to 3 weeks after birth. They showed an abnormal phenotype including a smaller size, a bigger head (FIG. 12) and also 100% penetrance (n=8) for dysplasia in the retina (FIG. 13).

To determine the stage of development affected by ASPP2 mutation, timed breedings followed by embryo genotyping were performed (table 1). Until birth, ASPP2 null embryos were viables and occurred at the expected Mendelian frequency. However, at the gross morphological level, null embryos showed severe brain malformations as early as embryonic day 11.5 (FIG. 14) and 37.5% exhibit neural tube defect like exencephaly and variant of craniorachischisis (FIG. 15A). In these mutants, the developing bones of the skull vault were missing while the base of the skull was present but deformed (FIG. 15B). These defects are probably secondary to the exencephalic development of the brain.

Expression of ASPP2 is Consistent with Brain Malformation During the Development.

The observed perinatal lethality indicates that ASPP2 plays an important role during development. To test this hypothesis, we first examined the expression of ASPP2 and one of its potential associating factor, p53, throughout development. RT-PCR of RNA isolated from different stages of WT embryos revealed an expression during the early development (FIG. 16A). In situ hybridization showed that ASPP2 is expressed from E9.5 in restricted areas, see FIG. 16B).

Cell Death and Proliferation in ASPP2 Null Embryos Brain?

Apoptosis is a prominent feature during early apoptosis of the nervous system. The retraction of the interdigital webbing in the fetal hand plate, a process involving apoptosis, is completed in mutant embryos by E13.5, according to the normal developmental schedule (data not shown).

ASPP2 Regulates the p53-Dependent Apoptosis in vivo?

p53 has been shown to be required for irradiation-induced apoptosis in the developing nervous system. To examine the requirement of ASPP2 in this setting, E13.5 control and ASPP2-null embryos were treated with -radiations (5 Gy) in utero, harvested 5 h later and analysed for apoptosis.

ASPP2 Regulates P53 in Tissues.

We then wished to assess the influence of the absence of one or two ASPP2 alleles on the latency and pathogenesis of neoplastic disease in p53-null or heterozygous mice. Crosses were performed using mice heterozygous for p53 and ASPP2 to produce the following cohorts: p53 (−/−)/ASPP2 (+/+), p53 (−/−)/ASPP2 (±), p53 (±)/ASPP2 (+/+), p53 (±)/ASPP2 (±) and p53 (±)/ASPP2 (−/−). To measure the tumour incidence, we plotted the number of animals that remained tumour-free against time, results not shown. Interestingly, the presence of one or two ASPP2 alleles did not interfere with the latency suggesting that ASPP2 is a upstream regulator of p53-induced apoptosis. However, the latency in the p53 (±)/ASPP2 (±) mice was significantly shorter than in p53 (±)/ASPP2 (+/+) mice.

We also performed a pathological examination of the tumours that arose in the p53 (−/−)/ASPP2 (+/+), p53 (−/−)/ASPP2 (±) and p53 (±)/ASPP2 (±) mice. Most tumours arising in the mice of each genotype were lymphomas, constituting approximately 80% of the tumours in p53(−/−) mice in the presence of either one or two alleles of ASPP2 (FIG. 17). They were all thymic in origin. Interestingly, the absence of one allele of ASPP2 slightly modified the spectrum of the other tumours.

The retinas of ASPP2 null mice also have an abnormal development as illustrated in FIG. 18. The phenotype is similar to retinoblastoma and represents a model for the study of this cancer.

TABLE 1
Genotyping of mice derived from ASPP2 heterozygous breeding.

| Genotype | +/+ | +/− | −/− | | Total |
|---|---|---|---|---|---|
| E9.5 | 4 | 13 | 4 | (20%) | 21 |
| E10.5 | 7 | 15 | 6 | (21%) | 28 |
| E11.5 | 5 | 14 | 6 | (25%) | 24 |
| E12.5 | 7 | 13 | 6 | (23%) | 26 |
| E13.5 | 17 | 23 | 24 | (37.5%) | 64 |
| E14.5 | | | | | |
| E15.5 | 3 | 4 | 4 | (36%) | 11 |
| E16.5 | | | | | |
| E17.5 | 4 | 10 | 6 | (30) | 20 |
| E18.5 | 0 | 5 | 3 | (37.5%) | 8 |
| P5-P20 | | | | (6.62%) | 600 |

1. (canceled)

2. A method for detecting a polypeptide in a cell or tissue sample, wherein the sample comprises a nerve cell or a nerve progenitor cell and wherein said polypeptide is selected from the group consisting of:
a) a polypeptide encoded by a nucleic acid molecule as represented by the sequence shown in SEQ ID NO: 8 or 9;
b) a polypeptide encoded by a nucleic acid molecule which hybridises to the nucleic acid molecule in (a); or
c) a polypeptide encoded by a nucleic acid molecule which is degenerate to the nucleic acid molecule represented in (a) and (b); said method comprising the steps of,
i) providing a sample comprising a nerve cell or a nerve cell progenitor cell;
ii) contacting said sample with an agent which binds said polypeptide; and
iii) detecting the presence of said polypeptide in said cell sample.

3. The method according to claim 2, wherein said polypeptide is encoded by a nucleic acid molecule which hybridises under stringent hybridisation conditions to the nucleic acid sequence as represented in SEQ ID NO: 8 or 9.

4. The method according to claim 3, wherein said nucleic acid is represented by the nucleic acid sequence in SEQ ID NO: 8 or 9.

5. The method of claim 2, wherein said polypeptide is represented by the amino acid sequence in SEQ ID NO: 10 or 11 wherein said sequence has been modified by addition, deletion or substitution of at least one amino acid residue.

6. The method of claim 2, wherein said agent is an antibody which binds said polypeptide.

7. The method according to claim 6 wherein said antibody is a polyclonal antibody or a monoclonal antibody.

8. (canceled)

9. The method of claim 6, wherein said antibody is provided with means which enable the detection of the antibody bound to said polypeptide.

10. The method according to claim 9, wherein said detection means is an enzyme; a isotope label or a fluorescent label.

11. The method of claim 1, wherein said method is the detection of a nucleic acid molecule which encodes said polypeptide.

12. The method according to claim 11, wherein said agent is a nucleic acid molecule adapted to anneal to said nucleic acid molecule which encodes said polypeptide.

13. The method according to claim 12, wherein said nucleic acid molecule is at least one oligonucleotide molecule.

14. The method according to claim 13, wherein said nucleic acid molecule is a pair of oligonucleotide molecules adapted to bind said nucleic acid molecule which is to be detected.

15. The method according to claim 14, wherein said method is a polymersase chain reaction method.

16. A method of treating neurodegenerative diseases which result from abnormal expression of a polypeptide, comprising administration of a medicament comprising the polypeptide, wherein the polypeptide is selected from the group consisting of:
i) a polypeptide encoded by a nucleic acid molecule as represented by the sequence shown in SEQ ID NO: 8 or 9;
ii) a polypeptide encoded by a nucleic acid molecule which hybridises to the nucleic acid molecule in (i); or
iii) a polypeptide encoded by a nucleic acid molecule which is degenerate because of the genetic code to the nucleic acid molecule represented in (i) and (ii).

17. The method according to claim 16, wherein said polypeptide is encoded by a nucleic acid molecule.

18. The method according to claim 16, wherein said polypeptide is represented by the amino acid sequence in SEQ ID NO: 10 or 11 wherein said sequence has been modified by addition, deletion or substitution of at least one amino acid residue.

19. The method according to claim 17 wherein said nucleic acid molecule is part of a vector adapted for gene therapy.

20. A method of treating neurodegenerative diseases which result from abnormal expression of a polypeptide, comprising administration of a medicament comprising an antagonist which interacts with the polypeptide, wherein the polypeptide is selected from the group consisting of:
i) a polypeptide encoded by a nucleic acid molecule as represented by the sequence in SEQ ID NO: 8 or 9;
ii) a polypeptide encoded by a nucleic acid molecule which hybridises to the nucleic acid molecule in (i); or
iii) a polypeptide encoded by a nucleic acid molecule which is degenerate to the nucleic acid molecule represented in (i) and (ii).

21. The method of claim 20, wherein said polypeptide is represented by the amino acid sequence in SEQ ID NO: 10 or 11 wherein said sequence has been modified by addition, deletion or substitution of at least one amino acid residue.

22. The method of claim 20, wherein said disease is Alzheimer's disease; Parkinson's disease; multiple sclerosis; or a retinopathy.

23. The method of claim 20, wherein said antagonist is an antibody or antibody part which binds said polypeptide.

24. The method of claim 23, wherein said antibody is a monoclonal antibody or binding part thereof.

25. The method of claim 23, wherein said antibody part is a Fab fragment.

26. The method of claim 25, wherein said antibody part is selected from the group consisting of: F(ab′)₂, Fab, Fv and Fd fragments; and CDR3 regions.

27. The method of claim 24, wherein said antibody is a humanised.

28. The method of claim 24, wherein said antibody is a chimeric antibody.

29. The method of claim 20, wherein said antagonist is a nucleic acid molecule.

30. The method of claim 29, wherein said nucleic acid molecule is a transcription cassette comprising an nucleic acid molecule operatively linked to a promoter which promoter transcribes said nucleic acid molecule to produce an antisense nucleic acid molecule, said sequence selected from the group consisting of:
i) a nucleic acid sequence, or part thereof, as represented in SEQ ID NO: 8 or 9;
ii) a nucleic acid sequence which hybridises to the sense sequence presented in SEQ ID NO: 8 or 9 and which encodes a polypeptide with anti-apoptotic activity.

31. The method of claim 30, wherein said cassette is part of a vector.

32. The method of claim 29, wherein said nucleic acid molecule comprises a transcription cassette wherein said a nucleic acid molecule, or part thereof, selected from the group consisting of:
i) a nucleic acid molecule represented by the nucleic acid sequence in SEQ ID NO:

8 or 9;
ii) a nucleic acid molecule which hybridises to the sequences in (i) above and which encodes a polypeptide with anti-apoptotic activity; or
iii) a nucleic acid molecule which is degenerate as a consequence of the genetic code to the sequences defined in (i) and/or (ii) above; wherein said cassette is adapted such that both sense and antisense nucleic acid molecules are transcribed from said cassette.

33. The method of claim 32, wherein said cassette is provided with at least two promoters adapted to transcribe both sense and antisense strands of said nucleic acid molecule.

34. The method of claim 32, wherein said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

35. The method of claim 34, wherein said first and second parts are linked by at least one nucleotide base.

36. The method of claim 35, wherein said first and second parts are linked by 2, 3, 4, 5, 6, 7, 8, 9 or at least 10 nucleotide bases.

37. The method of claim 32, wherein the length of said RNAi molecule is between 100 bp-1000 bp.

38. The method of claim 37, wherein the length of said RNAi molecule is at least 100 bp; 200 bp; 300 bp; 400 bp; 500 bp; 600 bp; 700 bp; 800 bp; 900 bp; or 1000 bp.

39. The method of claim 32, wherein said RNAi is at least 1000 bp in length.

40. The method of claim 32, wherein said RNAi molecule is between 15 bp and 25 bp in length.

41. The method of claim 40, wherein said RNAi molecule is 21 bp in length.

42. The method of claim 32 wherein said cassette is part of a vector.

43. A method to screen for agents which modulate the activity of a polypeptide which induces the apoptotic function of p53 comprising the steps of:
i) providing a cell sample comprising a nerve cell or nerve progenitor cell;
ii) contacting said sample with an agent to be tested; and
iii) monitoring effect of said agent on the presence and/or activity of said polypeptide.

44. The method according to claim 43, wherein said polypeptide is selected from the group consisting of:
a) a polypeptide encoded by a nucleic acid molecule as represented by the sequence shown in SEQ ID NO: 8 or 9;
b) a polypeptide encoded by a nucleic acid molecule which hybridises to the nucleic acid molecule in (a); or
c) a polypeptide encoded by a nucleic acid molecule which is degenerate to the nucleic acid molecule represented in (a) and (b).

45. The method according to claim 43 wherein said agent is an antagonist or agonist of said polypeptide.

46. (canceled)
